(19) United States
(12) Patent Application Publication
(10) Pub. No.: US 2025/0107972 A1
(43) Pub. Date: Apr. 3, 2025

(54) OIL-IN-WATER EMULSIONS CONTAINING UNALKOXYLATED WAX AND LATEX

(71) Applicant: L'OREAL, Paris (FR)

(72) Inventors: Travis VALADEZ, New York, NY (US); Diana FERNANDEZ, Edison, NJ (US); Susan DESTENO, Springfield, NJ (US); Marian GEORGES, East Brunswick, NJ (US)

(21) Appl. No.: 18/478,111

(22) Filed: Sep. 29, 2023

(51) Int. Cl. A61K 8/06; A61Q 1/10

(52) U.S. Cl. CPC A61K 8/062; A61Q 1/10; A61K 2800/30; A61K 2800/43

(57) ABSTRACT

The invention relates to oil-in-water (o/w) emulsion compositions comprising at least one latex and at least one unalkoxylated wax, in which the emulsion compositions preferably contain minimal amount of animal-derived ingredients and/or beeswax or are free of such ingredients, as well as to methods including such compositions.

### FIELD OF THE INVENTION

The present invention relates to oil-in-water (o/w) emulsion compositions comprising at least one latex and at least one unalkoxylated wax. Preferably, the o/w emulsion compositions contain minimal amounts of animal-derived ingredients or are free of such ingredients.

### DISCUSSION OF THE BACKGROUND

Many cosmetic compositions contain animal derived ingredients or natural beeswax to achieve a desired texture or desired properties in the compositions. Because of growing concerns over how such ingredients may be obtained, many customers prefer to avoid cosmetics containing such ingredients, opting instead for products not relying upon such ingredients.

However, without such ingredients, it can be difficult to obtain a desirable texture for the compositions, or it can be difficult to obtain other desirable properties such as, for example, volumization and tubing (removal from eyelashes in visible pieces) properties in a mascara composition.

Possibly relevant publications include:

U.S. patent application 2007/0212316 discloses mascara compositions containing a latex polymer, a self-emulsifying wax in an amount of at least 15% by weight of the composition, and water.

U.S. Pat. No. 9,687,426 discloses compositions containing an aqueous phase and an emulsifying system, where the emulsifying system contains at least one C₁₀-C₃₀ alkyl phosphate and at least one C₈-C₂₄ fatty alkyl ether of polyethylene glycol, the ether comprising from 1 to 19 ethylene glycol units and having an HLB<8 at 25° C.

Despite such references, there remains a need for improved cosmetic compositions having good properties, such as, for example, mascara compositions having good volumizing properties while maintaining good tubing (removal) properties, while at the same time preferably minimizing or eliminating the amount of animal-derived ingredients and/or other ingredients with perceived problematic production processes such as natural beeswax.

Accordingly, one aspect of the present invention is a cosmetic care and/or makeup and/or treatment composition, in particular for eyelashes, which has good volumizing and tubing (removal) properties while preferably minimizing or eliminating animal-derived ingredients and/or natural beeswax and providing for water and/or sebum resistance.

### SUMMARY OF THE INVENTION

The present invention relates to oil-in-water (o/w) emulsion compositions comprising water, at least one latex and at least one unalkoxylated wax, wherein the compositions contain a weight ratio of unalkoxylated wax to latex (active ingredient) of 1.0 or less. Preferably, the o/w emulsion compositions further comprise at least one aqueous phase film forming agent and/or at least one surfactant. Preferably, the o/w emulsion compositions are eyelash compositions, in particular mascara compositions which further comprise at least one coloring agent. Preferably, the o/w emulsion compositions contain minimal amounts of animal-derived ingredients or are free of such ingredients.

The present invention also relates to oil-in-water (o/w) emulsion compositions comprising water, at least one water-soluble natural gum, at least one latex and at least one unalkoxylated wax, wherein the compositions contain a weight ratio of unalkoxylated wax to latex (active ingredient) of 1.0 or less. Preferably, the o/w emulsion compositions further comprise at least one aqueous phase film forming agent and/or at least one surfactant. Preferably, the o/w emulsion compositions are eyelash compositions, in particular mascara compositions which further comprise at least one coloring agent. Preferably, the o/w emulsion compositions contain minimal amounts of animal-derived ingredients or are free of such ingredients.

The present invention also relates to methods of treating, caring for, enhancing the appearance of, and/or making up eyelashes comprising applying compositions of the present invention to eyelashes in an amount sufficient to treat, care for, enhance the appearance of, and/or make up the eyelashes.

The present invention also relates to methods of volumizing eyelashes comprising applying compositions of the present invention to eyelashes in an amount sufficient to volumize the eyelashes.

The present invention also relates to methods of improving removal properties of an eyelash composition from eyelashes in visible pieces comprising combining at least one latex and at least one unalkoxylated wax in a weight ratio of unalkoxylated wax to latex (active ingredient) of 1.0 or less, and water during formation of the emulsion compositions such that the eyelash composition can be removed from eyelashes in visible pieces during removal. Preferably, at least one aqueous phase film forming agent and/or at least one surfactant and/or at least one coloring agent and/or at least one water-soluble natural gum is also added during formation of the emulsion compositions. Preferably, the o/w emulsion compositions are mascara compositions which further comprise at least one coloring agent. Preferably, the o/w emulsion compositions contain minimal amounts of animal-derived ingredients or are free of such ingredients.

The present invention also relates to methods of making oil-in-water (o/w) emulsion compositions which are capable of volumizing eyelashes and/or tubing after application, the methods comprising combining at least one latex and at least one unalkoxylated wax in a weight ratio of unalkoxylated wax to latex (active ingredient) of 1.0 or less, and water during formation of the emulsion compositions. Preferably, the compositions possess sebum and/or water-resistance properties. Preferably, at least one aqueous phase film forming agent and/or at least one surfactant and/or at least one coloring agent and/or at least one water-soluble natural gum is also added during formation of the emulsion compositions. Preferably, the o/w emulsion compositions are eyelash compositions, in particular mascara compositions which further comprise at least one coloring agent. Preferably, the o/w emulsion compositions contain minimal amounts of animal-derived ingredients or are free of such ingredients.

It is to be understood that both the foregoing general description and the following detailed description are exemplary and explanatory only, and are not restrictive of the invention.

### DETAILED DESCRIPTION OF THE INVENTION

In the following description of the invention and the claims appended hereto, it is to be understood that the terms used have their ordinary and accustomed meanings in the art, unless otherwise specified.

“About” as used herein means within 10% of the indicated number (e.g., “about 10%” means 9%-11% and “about 2%” means 1.8%-2.2%).

“A” or “an” as used herein means “at least one.”

“At least one” means one or more and thus includes individual components as well as mixtures/combinations.

As used herein, all ranges provided are meant to include every specific range within, and combination of subranges between, the given ranges. Thus, a range from 1-5, includes specifically 1, 2, 3, 4 and 5, as well as subranges such as and 2-5, 3-5, 2-3, 2-4, 1-4, etc.

“Film former”, “film-forming polymer” or “film-forming agent” as used herein means a polymer or resin that leaves a film on the substrate to which it is applied, for example, after a solvent accompanying the film former has evaporated, absorbed into and/or dissipated on the substrate.

“Wax” as used herein is a lipophilic fatty compound that is solid at ambient temperature (25° C.) and changes from the solid to the liquid state reversibly, having a melting temperature of more than 30° C. and, for example, more than 45° C., and a hardness of more than 0.5 MPa at ambient temperature.

“Surfactant” and “emulsifier” are used interchangeably throughout this specification.

“Substituted” as used herein, means comprising at least one substituent. Non-limiting examples of substituents include atoms, such as oxygen atoms and nitrogen atoms, as well as functional groups, such as hydroxyl groups, ether groups, alkoxy groups, acyloxyalky groups, oxyalkylene groups, polyoxyalkylene groups, carboxylic acid groups, amine groups, acylamino groups, amide groups, halogen containing groups, ester groups, thiol groups, sulphonate groups, thiosulphate groups, siloxane groups, and polysiloxane groups. The substituent(s) may be further substituted.

“Volatile”, as used herein, means having a flash point of less than about 115° C.

“Non-volatile”, as used herein, means having a flash point of greater than about 115° C.

“Polymer” as used herein means a compound which is made up of at least two monomers.

“Free” or “substantially free” or “devoid of” as it is used herein means that while it is preferred that no amount of the specific component be present in the composition, it is possible to have very small amounts of it in the compositions of the invention provided that these amounts do not materially affect at least one, preferably most, of the advantageous properties of the compositions of the invention. Thus, for example, “free of animal-derived ingredients” means that an effective amount (that is, more than trace amounts) of animal-derived ingredient(s) is/are omitted from the composition (that is, about 0% by weight), “substantially free of animal-derived ingredients” means that animal-derived ingredient(s) is/are present in amounts not greater than 0.1% by weight, and “devoid of animal-derived ingredients” means that animal-derived ingredient(s) is/are present in amounts not greater than 0.25% by weight, based on the total weight of the composition. The same nomenclature applies for all other ingredients identified throughout the application and in this paragraph such as, for example, silicone oils (compositions of the invention which are “free of silicone oils,” “substantially free of silicone oils,” and “devoid of silicone oils” have meanings consistent with the discussion within this paragraph), even if not specifically discussed for each identified ingredient in the application. Discussed examples of the use of such language such as those in this paragraph are intended to be exemplary, not limiting.

“Makeup Result” as used herein, refers to compositions where color remains the same or substantially the same as at the time of application, as viewed by the naked eye, after an extended period of time. “Makeup Result” may be evaluated by evaluating long wear properties by any method known in the art for evaluating such properties. For example, long wear may be evaluated by a test involving the application of a composition to keratin materials such as eyelashes and evaluating the color of the composition after an extended period of time. For example, the color of a composition may be evaluated immediately following application to keratin materials such as eyelashes and these characteristics may then be re-evaluated and compared after a certain amount of time. Further, these characteristics may be evaluated with respect to other compositions, such as commercially available compositions.

“Keratinous material” or “keratin material” means natural nails, lips, skin such as the face, the body, the hands, and the area around the eyes, and keratin fibres such as head hair, eyelashes, eyebrows, bodily hair of a human, as well as synthetic additions such as false eyelashes, false eyebrows, false nails, etc.

“Physiologically acceptable” means compatible with eyelashes and having a pleasant color, odor and feel, and which does not cause any unacceptable discomfort (stinging or tautness) liable to discourage a consumer from using the composition.

“Water-soluble” refers to solubility of a compound in water at room temperature or at temperatures at which water emerges from a typical faucet.

“Natural” as in the phrase “natural compound” or “natural gum” refers to any compound derived directly from a natural substance such as a plant without having undergone any chemical modification.

“Compound (e.g., wax) of natural origin” refers to any compound derived from a natural compound which has undergone one or more chemical modifications, for example by organic synthesis reaction, without the properties of the natural compound having been modified.

“Synthetic compound” refers to any compound which is not a natural compound or a compound of natural origin.

“Room temperature” means 25° C.

“Atmospheric pressure” means 760 mmHg, i.e. 105 pascals.

“Tubing” means removal properties of an eyelash composition from eyelashes in visible pieces.

The compositions and methods of the present invention can comprise, consist of, or consist essentially of the essential elements and limitations of the invention described herein, as well as any additional or optional ingredients, components, or limitations described herein or otherwise useful. For example, the aqueous phase solvent system of the oil-in-water emulsion compositions of the invention can “consist essentially of” water and optionally at least one C2-C5 monoalcohol and/or at least one humectant (specifically, e.g., polyol).

For purposes of the present invention, the “basic and novel property” associated with compositions, components and methods which “consist essentially of” identified ingredients or actions is “both volumization upon application to eyelashes and tubing during removal from eyelashes.”

Referred to herein are trade names for materials including, but not limited to polymers and optional components. The inventors herein do not intend to be limited by materials described and referenced by a certain trade name. Equivalent materials (e.g., those obtained from a different source under a different name or catalog (reference) number) to those referenced by trade name may be substituted and utilized in the methods described and claimed herein.

All percentages and ratios are calculated by weight unless otherwise indicated. All percentages are calculated based on the total weight of a composition unless otherwise indicated. All component or composition levels are in reference to the active level of that component or composition, and are exclusive of impurities, for example, residual solvents or by-products, which may be present in commercially available sources.

All U.S. patents or patent applications disclosed herein are expressly incorporated by reference in their entirety.

### Latex

According to the present invention, o/w emulsion compositions comprising at least one latex are provided. “Latex” is a colloidal dispersion of polymer particles in an aqueous liquid phase. “Latex” is generally obtained by suspension or emulsion polymerization or copolymerization of monomers according to processes that are well known to those of ordinary skill in the art. Such monomers may be chosen in particular from styrene, butadiene, acrylonitrile, chloroprene, vinyl acetate, urethanes, isoprene, isobutylene, and acrylic or methacrylic acid, maleic acid, crotonic acid or itaconic acid or esters or amides thereof.

Suitable latexes include particles of polymers comprising at least one styrene group. Preferably, such latexes have molecular weights of more than 100,000 Da weight average molecular weight, preferably more than 120,000 Da, preferably more than 135,000 Da, and preferably more than 150,000 Da, including all ranges and subranges therebetween.

Any styrene acrylate copolymer or derivative thereof may be suitable for use in the compositions of the present invention. The latex comprising at least one styrene group preferably has a glass transition temperature (Tg) ranging from about −15° C. to about 90° C., such as from about 0° C. to about 50° C., including all ranges and subranges therebetween. For example, a copolymer comprising (1) styrene and ammonium acrylates, (2) acrylates, and/or (3) acrylic groups are suitable. A particularly preferred copolymer is a styrene/acrylates/ammonium methacrylate copolymer or a styrene acrylic copolymer, commercial examples of which include SYNTRAN 5620 and SYNTRAN 5760 (with or without paraben), by Interpolymer Corporation; JONCRYL 77, by BASF Performance Chemicals; and RHOPLEX P376, by Dow Chemical Company.

Suitable latexes also include latexes which lack styrene groups. According to certain embodiments, the latex lacking styrene groups are copolymers comprising two or more monomers chosen from acrylic acid, methacrylic acid, and their simple esters, for example, lower alkyl esters such as methyl, ethyl, and ethylhexyl esters. For example, copolymers may be chosen from acrylates copolymers, ammonium acrylates copolymers, ethyl acrylates copolymers, acrylates/ethylhexylacrylate copolymers, acrylates/octylacrylates copolymers, alkyl (meth)acrylates copolymers, acrylates/C₁₂-C₂₂ alkylmethacrylate copolymers, ethylacrylate/methacrylic acid copolymer, ethylhexyl acrylate/butyl acrylate/methacrylic acid, methyl methacrylate/butyl acrylate/methacrylic acid and t-butyl acrylate/ethyl acrylate/methacrylic acid copolymer. Examples of commercially available acrylate copolymers include, but are not limited to, DAITOSOL 3000 SLPN, DAITOSOL 4000 SJT, DAITOSOL 5000 AD, DAITOSOL 5000 SJ, KOBOGUARD® 50A, and KOBOGUARD® 50N sold by Kobo Products, Inc.

Preferably, the at least one latex is present in the emulsion compositions of the present invention in an amount of active material (that is, an amount not including the aqueous liquid phase of the latex) of from about 1% to about 30%, preferably from about 4% to about 25%, and preferably from about 7.5% to about 15% by weight, based on the total weight of the composition, including all ranges and subranges in between, such as from 5% to 20%, 10% to 20%, 8% to 18%, etc.

### Unalkoxylated Wax

According to the present invention, o/w emulsion compositions comprising at least one unalkoxylated wax are provided. “Unalkoxylated” means that the wax is not self-emulsifying: for example, the wax does not contain sufficient alkoxylated groups such as polyethyleneglycol (poly(oxyethylene)), polypropyleneglycol (poly(oxypropylene)), etc., to provide the wax with self-emulsifying properties. Preferably, unalkoxylated waxes are free of alkoxylated groups. Preferably, the at least one unalkoxylated wax has a melting point of at least 40° C., preferably at least 50° C., and preferably at least 60° C.

Generally speaking, unalkoxylated waxes suitable for use in the invention w/o emulsion compositions are natural waxes and/or waxes of natural origin and/or straight chain fatty acid or fatty alcohol compounds, and/or mixtures thereof, preferably containing between 8 and 40 carbon atoms, preferably between 16 and 36 carbon atoms. For example, synthentic beeswax, which is mainly a mixture of esters of even-numbered, straight chain acids and alcohols containing 16-36 carbon atoms, is preferred for use in the invention compositions. Such synthetic beeswax is commercially available, for example, from Evonik Goldschmidt under the name Cyclochem 330.

Other examples include fatty alcohol waxes containing from about 8 to about 30 carbon atoms, preferably from about 12 to about 28 carbon atoms, and preferably from about 14 to about 24 carbon atoms, including but not limited to 8 to 24 carbon atoms, 12 to 16 carbon atoms, 16 to 22 carbon atoms, etc. The fatty alcohol wax can be saturated or unsaturated, linear or branched. Mention may be made, for example, of cetyl alcohol, stearyl alcohol, behenyl alcohol, and mixtures thereof. Other examples of suitable fatty alcohol waxes include waxes including natural components (such as from one or more natural oils) and fatty alcohol components such as, for example, hydrogenated stearyl olive ester, commercially available from the supplier Sophim under the tradename Phytowax Olive 18 L 57, and hydrogenated myristyl olive ester, commercially available from the supplier Sophim under the tradename Phytowax Olive 14 L 48.

Suitable examples of natural waxes that can be used in accordance with the present disclosure include those generally used in the cosmetics field: they include waxes such as carnauba wax, candelilla wax, ouricoury wax, Japan wax, sunflower seed wax, cork fibre wax or sugar cane wax, rice wax, montan wax, paraffin wax, lignite wax or microcrystalline wax, ceresin, ozokerite and jojoba butter. Carnauba wax and jojoba butter are preferred natural waxes.

Preferably, however, the oil phase of the o/w emulsion composition contains less than 5% by weight of natural beeswax, preferably less than 2.5% by weight of natural beeswax, preferably less than 1% by weight of natural beeswax, or preferably is “substantially free,” “devoid” of or “free” of natural beeswax as defined above.

Preferably, however, the oil phase of the o/w emulsion composition contains less than 5% by weight of synethetic wax(es) (other than synthetic beeswax), preferably less than 2.5% by weight of synethetic wax(es), preferably less than 1% by weight of synethetic wax(es), or preferably is “substantially free,” “devoid” of or “free” of synethetic wax(es) as defined above.

Preferably, however, the oil phase of the o/w emulsion composition contains less than 5% by weight of silicone wax(es), preferably less than 2.5% by weight of silicone wax(es), preferably less than 1% by weight of silicone wax(es), or preferably is “substantially free,” “devoid” of or “free” of silicone wax(es) as defined above.

Preferably, however, the oil phase of the o/w emulsion composition contains less than 5% by weight of paraffin wax, preferably less than 2.5% by weight of paraffin wax, preferably less than 1% by weight of paraffin wax, or preferably is “substantially free,” “devoid” of or “free” of paraffin wax as defined above.

Preferably, the at least one unalkoxylated wax is present in an amount of from about 1% to 25% by weight, preferably from about 3% to about 20% by weight, preferably from about 5% to about 15% by weight, and preferably from about 6% to about 12% by weight relative to the total weight of the composition, including all ranges and subranges therebetween, including for example, 1% to 17%, 5% to 20%, 7% to 15%, etc.

According to preferred embodiments, the unalkoxylated wax and latex are present in compositions of the present invention in a weight ratio of unalkoxylated wax to latex (active ingredient) of 1.0 or less, preferably 0.95 or less, preferably 0.9 or less and preferably 0.85 or less, including all ranges and subranges encompassed within these ratios as long as more latex (active ingredient) is present in the composition than unalkoxylated wax.

### Water/Aqueous Phase Solvent System

According to the present invention, compositions comprising an aqueous phase solvent system comprising (i) water in an amount of at least about 5% by weight with respect to the total weight of the composition; (ii) optionally at least one C2-C5 monoalcohol; and (iii) optionally at least one humectant are provided.

According to the present invention, the compositions comprise water. Preferably, the compositions comprise from about 10% to about 60% water by weight, preferably from about 15% to about 55% by weight, preferably from about 20% to less than 50% by weight, preferably from about 25% to less than 50% by weight, and preferably from about 30% to about 45% by weight of water, all weights being based on the total weight of the composition, including all ranges and subranges therebetween such as, for example, about 15% to less than 50%.

According to preferred embodiments, the compositions may further comprise at least one monoalcohol. Suitable C2-C5 monoalcohols include, for example, ethanol, propanol, butanol, pentanol, isopropanol, isobutanol and isopentanol. Ethanol is particularly preferred.

If present, the C2-C5 monoalcohol(s) is/are preferably present in the compositions of the present invention in an amount ranging from about 1% to about 20% by weight, preferably from about 2.5% to about 15% by weight, preferably from about 3% to about 10% by weight, and preferably from about 4% to about 8%, by weight, all weights being based on the total weight of the composition, including all ranges and subranges in between such as, for example, about 3% to about 10%.

Preferably, more water is present in the compositions of the present invention than C2-C5 monoalcohol.

According to preferred embodiments, compositions of the present invention optionally further comprise at least one humectant. Preferably, the at least one humectant comprises at least one hydroxy group or is a derivative of a compound comprising at least one hydroxy group. Suitable humectants of this type are known in the art and include, but are not limited to, polyols. Specific suitable humectants include, for example, propylene glycol, butylene glycol, hexylene glycol, glycerine (sometimes known as glycerol or glycerin) and glucose ethers. Preferably, the at least one humectant is selected from glycerine, propylene glycol, butylene glycol, and mixtures thereof.

If present, the at least one humectant is present in the compositions of the present invention in an amount ranging from about 0.1% to about 10% by weight, preferably from about 0.25% to about 6% by weight, preferably from about 0.5% to about 5% by weight, and preferably from about 0.5% to about 2.5% by weight with respect to the weight of the composition, including all ranges and subranges therebetween such as, for example, 1% to 10%, 0.5% to 7.5%, 0.1% to 2.5%, 1% to 5%, etc.

### Oil/Oil Phase Solvent System

According to certain embodiments of the present invention, o/w emulsion compositions optional comprising at least one oil are provided.

Suitable oils include natural oils (discussed separately below), as well as other volatile and/or non-volatile, silicone and/or hydrocarbon oils.

Preferably, however, the oil phase of the o/w compositions of the invention contains less than 10% by weight of silicone oil(s), preferably less than 5% by weight of silicone oil(s), preferably less than 2.5% by weight of silicone oil(s), or preferably is “substantially free,” “devoid” of or “free” of silicone oil(s) as defined above.

Preferably, however, the oil phase of the o/w compositions of the invention contains less than 10% by weight of volatile oil(s), preferably less than 5% by weight of volatile oil(s), preferably less than 2.5% by weight of volatile oil(s), or preferably is “substantially free,” “devoid” of or “free” of volatile oil(s) as defined above.

According to certain embodiments, the compositions of the present invention may comprise one or more volatile silicone oils. Examples of such volatile silicone oils include linear or cyclic silicone oils having a viscosity at room temperature less than or equal to 6 cSt and having from 2 to 7 silicon atoms, these silicones being optionally substituted with alkyl or alkoxy groups of 1 to 10 carbon atoms. Specific oils that may be used in the invention include octamethyltetrasiloxane, decamethylcyclopentasiloxane, dodecamethylcyclohexasiloxane, heptamethyloctyltrisiloxane, hexamethyldisiloxane, decamethyltetrasiloxane, dodecamethylpentasiloxane and their mixtures. Other volatile oils which may be used include KF 96A of 6 cSt viscosity, a commercial product from Shin Etsu having a flash point of 94° C. Preferably, the volatile silicone oils have a flash point of at least 40° C.

Non-limiting examples of volatile silicone oils are listed in Table 1 below.

TABLE 1

| | Flash Point | Viscosity |
|---|---|---|
| Compound | (° C.) | (cSt) |
| Octyltrimethicone | 93 | 1.2 |
| Hexyltrimethicone | 79 | 1.2 |
| Decamethylcyclopentasiloxane | 72 | 4.2 |
| (cyclopentasiloxane or D5) | | |
| Octamethylcyclotetrasiloxane | 55 | 2.5 |
| (cyclotetradimethylsiloxane or D4) | | |
| Dodecamethylcyclohexasiloxane (D6) | 93 | 7 |
| Decamethyltetrasiloxane(L4) | 63 | 1.7 |
| KF-96 A from Shin Etsu | 94 | 6 |
| PDMS (polydimethylsiloxane) DC 200 | 56 | 1.5 |
| (1.5 cSt) from Dow Corning | | |
| PDMS DC 200 (2 cSt) from Dow Corning | 87 | 2 |

Further, a volatile linear silicone oil may be employed in the present invention. Suitable volatile linear silicone oils include those described in U.S. Pat. No. 6,338,839 and WO03/042221, the contents of which are incorporated herein by reference. In one embodiment the volatile linear silicone oil is decamethyltetrasiloxane. In another embodiment, the decamethyltetrasiloxane is further combined with another solvent that is more volatile than decamethyltetrasiloxane.

According to certain embodiments of the present invention, the composition of may comprise one or more non-silicone volatile oils and may be selected from volatile hydrocarbon oils, volatile esters and volatile ethers. Examples of such volatile non-silicone oils include, but are not limited to, volatile hydrocarbon oils having from 8 to 16 carbon atoms and their mixtures and in particular branched C₈ to C₁₆ alkanes such as C₈ to C₁₆ isoalkanes (also known as isoparaffins), isohexacecane, isododecane, isodecane, and for example, the oils sold under the trade names of Isopar or Permethyl. Preferably, the volatile non-silicone oils have a flash point of at least 40° C.

Non-limiting examples of volatile non-silicone volatile oils are given in Table 2 below.

TABLE 2

| Compound | Flash Point (° C.) |
|---|---|
| sododecane | 43 |
| Propylene glycol n-butyl ether | 60 |
| Ethyl 3-ethoxypropionate | 58 |
| Propylene glycol methylether acetate | 46 |
| Isopar L (isoparaffin C₁₁-C₁₃) | 62 |
| Isopar H (isoparaffin C₁₁-C₁₂) | 56 |

The volatility of the solvents/oils can be determined using the evaporation speed as set forth in U.S. Pat. No. 6,338,839, the contents of which are incorporated by reference herein.

According to certain embodiments of the present invention, the composition may comprise at least one non-volatile oil which is not a natural oil (discussed separately below). Examples of non-volatile oils that may be used in the present invention include, but are not limited to, polar oils such as, for example:
caprylic/capric acid triglycerides, for instance those sold by the company Stearineries Dubois or those sold under the names Miglyol 810, 812 and 818 by the company Dynamit Nobel;
oils or esters of formula R₅COOR₆ in which R₅ represents a linear or branched higher fatty acid residue containing from 1 to 40 carbon atoms, including from 7 to 19 carbon atoms, and R₆ represents a branched hydrocarbon-based chain containing from 1 to 40 carbon atoms, including from 3 to 20 carbon atoms, with R₆+R₇≥10, such as, for example, Purcellin oil (cetostearyl octanoate), isononyl isononanoate, octyldodecyl neopentanoate, C₁₂ to C₁₅ alkyl benzoate, isopropyl myristate, 2-ethylhexyl palmitate, and octanoates, decanoates or ricinoleates of alcohols or of polyalcohols; hydroxylated esters, for instance isostearyl lactate or diisostearyl malate; and pentaerythritol esters;
ethers containing from 10 to 40 carbon atoms;
liquid C₈ to C₂₆ fatty alcohols, for instance oleyl alcohol, cetyl alcohol, stearyl alcohol, octyldodecanol, and cetearly alcohol;
esters having melting points of more than 25° C. and less than 40° C. which function as additional oils, such as bis-diglyceryl polyacyladipate; and
mixtures thereof. Preferred additional oils are esters having melting points of more than 25° C. and less than 40° C., such as bis-diglyceryl polyacyladipate. Although not wishing to be bound by any theory, it is believed that inclusion of such ingredients surprisingly increases hardness of the resulting compositions when combined with the other ingredients of the present invention. Also preferred are liquid C₈ to C₂₆ fatty alcohols such as octyldodecanol.

Further, examples of non-volatile oils that may be used in the present invention include, but are not limited to, non-polar oils such as branched and unbranched hydrocarbons and hydrocarbon waxes including polyolefins, in particular Vaseline (petrolatum), paraffin oil, squalane, squalene, hydrogenated polyisobutene, hydrogenated polydecene, polybutene, mineral oil, pentahydrosqualene, and mixtures thereof.

According to certain embodiments of the present invention, the compositions of the present invention may comprise at least one non-volatile silicone oil. Suitable examples of such silicone oils include, but are not limited to, non-volatile silicone fluids such as, for example, polyalkyl (aryl) siloxanes. Suitable polyalkyl siloxanes include, but are not limited to, polydimethyl siloxanes, which have the CTFA designation dimethicone, polydiethyl siloxane, phenyl trimethicone, trimethyl pentaphenyl trisiloxane, phenyldimethicone, phenyltrimethylsiloxydiphenylsiloxane, diphenyldimethicone, and diphenylmethyldiphenyltrisiloxane and those siloxanes disclosed in U.S. patent application publication no. 2004/0126350, the entire disclosure of which is hereby incorporated by reference. Specific examples of suitable high viscosity silicone oils include, but are not limited to, 15 M 30 from PCR (500 cSt) or Belsil PDM 1000 (1 000 cSt) from Wacker and Dow Corning 200 (350 cSt) (the values in parenthesis represent viscosities at 25° C.).

According to preferred embodiments, the at least one oil, if present, is present in the compositions of the present invention in an amount ranging from about 0.1% to about 20% by weight, more preferably from about 0.25 to about 10% by weight, and preferably from about 0.5% to about 5% by weight, based on the total weight of the composition, including all ranges and subranges within these ranges such as, for example, 0.1% to 2%, 1% to 3%, etc.

### Natural Oil

According to preferred embodiments, o/w emulsion compositions may optionally further comprise at least one natural oil. The natural oil(s) are oils recovered or extracted from foods, and/or preferably oils recovered from plants or other vegetative life (as opposed to oils which are derived from natual sources through reactions and which are not “natural oil(s)” as used herein). For example, plant oils include glyceride triesters, which are generally triesters of fatty acids and of glycerol, the fatty acids of which can have varied chain lengths from C4 to C24, it being possible for these chains to be saturated or unsaturated and linear or branched.

Non-limiting examples of suitable natural oil(s) include olive oil, sweet almond oil, coconut oil, avocado oil, wheat germ oil, sunflower oil, grape seed oil, sesame oil, corn oil, apricot oil, castor oil, shea oil, soybean oil, palm oil, rapeseed oil, cottonseed oil, hazelnut oil, macadamia oil, jojoba oil, alfalfa oil, poppy oil, pumpkin seed oil, marrow oil, blackcurrant oil, evening primrose oil, millet oil, barley oil, quinoa oil, rye oil, safflower oil, candlenut oil, passionflower oil, musk rose oil, etc., including oils from seeds of the above, and including mixtures thereof. Castor (seed) oil is particularly preferred.

Preferably, if present, the at least one natural oil is present in an amount of from about 0.1% to 20% by weight, preferably from about 0.25% to about 15% by weight, preferably from about 0.5% to about 10% by weight, and preferably from about 1% to about 5% by weight relative to the total weight of the composition, including all ranges and subranges therebetween.

### Water-Soluble Natural Gum

According to preferred embodiments, o/w emulsion compositions of the present invention may optionally further comprise at least one water-soluble natural gum. Preferred water-soluble natural gums include, but are not limited to, guar, acacia (Senegal) gum, xanthan, gum arabic, gum tragacanth, locust bean bum, agar, and mixtures thereof. Acacia (Senegal) gum is preferred.

Although not wishing to be bound by any theory, it is believed that during the process of removing compositions of the present invention from eyelashes with water, the at least one water-soluble natural gum solubilizes sufficiently to disrupt the structure of the composition applied to eyelashes such that the composition can be more easily removed. As such, the at least one water-soluble natural gum, if present, is preferably present in an amount sufficient to increase removal properties of compositions of the present invention which have been applied to eyelashes (and allowed to dry) after exposure to water during the removal process.

Preferably, if present, the at least one water-soluble natural gum is present in an amount of from about 0.1% to 20% by weight, preferably from about 0.5% to about 10% by weight, preferably from about 0.75% to about 7.5% by weight, and preferably from about 1% to about 5% by weight relative to the total weight of the composition, including all ranges and subranges therebetween.

### Coloring Agent (Colorant)

According to preferred embodiments of the present invention, o/w emulsion compositions optionally further comprising at least one coloring agent are provided.

According to preferred embodiments, the at least one coloring agent is preferably chosen from pigments, dyes, such as liposoluble dyes, nacreous pigments, and pearling agents.

Representative liposoluble dyes which may be used according to the present invention include Sudan Red, DC Red 17, DC Green 6, ßne, soybean oil, Sudan Brown, DC Yellow 11, DC Violet 2, DC Orange 5, annatto, and quinoline yellow.

The nacreous pigments which may be used according to the present invention may be chosen from white nacreous pigments such as mica coated with titanium or with bismuth oxychloride, colored nacreous pigments such as titanium mica with iron oxides, titanium mica with ferric blue or chromium oxide, titanium mica with an organic pigment chosen from those mentioned above, and nacreous pigments based on bismuth oxychloride.

The pigments, which may be used according to the present invention, may be chosen from white, colored, inorganic, organic, polymeric, nonpolymeric, coated and uncoated pigments. Representative examples of mineral pigments include titanium dioxide, optionally surface-treated, surface-treated mica, zirconium oxide, zinc oxide, cerium oxide, iron oxides, chromium oxides, manganese violet, ultramarine blue, chromium hydrate, and ferric blue. Representative examples of organic pigments include carbon black, pigments of D & C type, and lakes based on cochineal carmine, barium, strontium, calcium, and aluminum.

If present, the coloring agent(s) Is/are present in an amount of from about 1% to 30% by weight, preferably from about 1% to about 20% by weight, preferably from about 3 to about 15%, and preferably from about 5% to about 15% by weight relative to the total weight of the composition, including all ranges and subranges therebetween, including for example, 6% to 16%, 2.5% to 18% 10% to 20%, etc.

If sufficient coloring agent(s) is/are present in the composition to provide color upon application to keratinous material (e.g., eyelashes), then the composition is preferably used at least as a cosmetic composition for affecting the color appearance of the eyelashes. If insufficient coloring agent(s) is/are present to provide color after application to eyelashes, then the composition is preferably used at least as a cosmetic composition for aspects other than affecting the appearance of the eyelashes such as a topcoat or primer composition.

### Surfactant

According to preferred embodiments of the present invention, compositions further comprising at least one surfactant are provided. Preferably, the at least one surfactant is a high HLB surfactant.

“HLB” refers to the “hydrophilic-lipophilic balance” associated with emulsifiers/surfactants. In particular, “HLB” value relates to the ratio of hydrophilic groups and lipophilic groups in emulsifiers, and also relates to solubility of the emulsifiers. Lower HLB emulsifiers are more soluble in oils (lipophilic material) and are more appropriate for use in water-in-oil (W/O) emulsions. Higher HLB emulsifiers are more soluble in water (hydrophilic material) and are more appropriate for oil-in-water (O/W) emulsions. In the context of the present invention, “high HLB surfactant” means a surfactant having an HLB value of greater than 8. Also, in the context of the present invention, “high HLB value” means a surfactant having an HLB value greater than or equal to 8, preferably greater than or equal to 9, preferably greater than or equal to 10, preferably greater than or equal to 12, and preferably greater than or equal to 13.

Examples of surfactants include, but are not limited to, the following:

Polyethylene glycolated (PEG) and/or glycerolated esters or ethers, such as polyethylene glycolated and/or glycerolated branched or linear C8-C24 compounds, for example, Oleth-50, Oleth-20, Oleth-10, Ceteth-10, Steareth-20, Laureth-23, PEG-8 stearate, PEG-8 isostearate, PEG-20 stearate PEG-40 stearate, etc.;

sorbitan esters or ethers, such as oxyethylenated or nonoxyethylenated sorbitan mono- or polyalkyl esters or ethers, such as Polysorbate 21, Polysorbate 40, Polysorbate 80, Polysorbate 60, Polysorbate 61, sorbitan isostearate, glyceryl sorbitan isostearate, sorbitan sesquioleate, sorbitan laurate, sorbitan monopalmitate, sorbitan oleate, sorbitan trioleate, sorbitan monostearate and sorbitan tristearate, etc.

Alkoxylated alkenyl succinates; and

Hydrophilically-modified silicone compounds such as, for example, polyethylene glycolated (PEG), polypropylene glycolaged (PPG) and/or glycerolated esters or ethers of silicone compounds, such as polyethylene glycolated and/or polypropylene glycolated and/or glycerolated branched or linear dimethicones such as for example, dimethicone copolyols, for example the mixture of cyclomethicone and of dimethicone copolyol sold under the name “DC 5225 C” by Dow Corning; alkyl dimethicone copolyols, such as lauryl methicone copolyol, sold under the name “Dow Corning 5200 Formulation Aid” by Dow Corning, and cetyl dimethicone copolyol, sold under the name “Abil EM 90” by Goldschmidt; the polyglyceryl-4 isostearate/cetyl dimethicone copolyol/hexyl laurate mixture sold under the name “Abil WE 90” by Goldschmidt; and PEG and/or PPG modified dimethicones such as PEG/PPG 17/18 dimethicone.

The at least one surfactant is preferably present in the compositions of the present invention in an amount of about 0.1% to about 15% by weight, preferably from about 1% to about 12% by weight, and preferably from about 3% to about 10% by weight with respect to the total weight of the composition, including all ranges and subranges therebetween.

### Aqueous Phase Film Forming Agent

According to preferred embodiments of the present invention, compositions of the present invention may further comprise at least one aqueous phase film-forming agent that is non-ionic water-soluble or water dispersible film forming agent or polymer. For clarity, by “non-ionic water-soluble or water-dispersible” it is meant that the film former is non-ionic. The polymer is also water-soluble or water-dispersible, particularly to the extent that it can be readily stabilized throughout a vehicle (e.g., water) present in the composition.

The at least one aqueous phase film forming agent is part of a aqueous phase film-forming system. The aqueous phase film forming system can comprise, consist essentially of, or consist of the at least one aqueous phase film former.

Examples of aqueous phase film forming agents include, but are not limited to, homopolymers and copolymers containing at least one (meth)acrylic acid ((meth)acrylate) monomer and/or at least one vinylpyrrolidone monomer such as polyvinylpyrrolidone, vinyl pyrrolidone/vinyl acetate copolymers, vinyl pyrrolidone/acrylic acid copolymers, vinyl pyrrolidone/acrylate copolymers, alkyl (e.g., butyl) acrylate/hydroxyalkyl (e.g., propyl) dimethicone acrylate copolymers, acrylamide/sodium acryloyldimethyltaurate copolymer, etc.; water-soluble polysaccharides such as pectin, unmodified pullulan or cellulose compounds; and mixtures thereof.

According to preferred embodiments, the at least one aqueous phase film forming agent is at least one cyclic amide-containing polymer. Cyclic amide monomers useful in the preferred polymers include those having one or more aromatic or aliphatic ring structures. These rings may have sizes ranging from about having sizes of, for example, 5 to 8 ring members.

In certain embodiments, monomers useful in forming these film-forming polymers are polymerizable, ethylenically-unsaturated monomers having a cyclic amide residue. Accordingly, the cyclic amide monomers of these film-forming polymers may include cyclic amide residues that are or include heterocyclic ring structures such as lactams and the like. These may include α-Lactam, β-lactam, γ-lactam, δ-lactam, and ε-lactam. In one preferred embodiment, the cyclic amide is a pyrrolidone (a γ-lactam), in particular vinylpyrrolidone. A particularly preferred polymer is a VP copolymer.

Preferred film formers include polymer(s) containing cyclic amide residue(s) such as, for example, vinylpyrrolidone homopolymers (polyvinylpyrrolidone) and vinylpyrrolidone copolymers containing at least one monomer other than vinylpyrrolidone such as, for example, substituted or unsubstituted C2 (acrylic acid) or C3 (allyl) groups, possibly in the form of esters (for example, acrylates or methacrylates) or ethers. VP/eicosane is particularly preferred.

In certain embodiments, the at least one aqueous phase film forming agent has a weight average molecular weight in a range from about 10,000 daltons to about 1,000,000 daltons, including all ranges and subranges therebetween such as, for example, 50,000 daltons to 500,000 daltons, 75,000 daltons to 300,000 daltons, and 100,000 daltons to 250,000 daltons.

Preferably, if present, the at least one aqueous phase film forming agent is present in the compositions of the present invention in an amount ranging from about 0.1% to about 10% by weight, preferably from about 0.2% to about 5% by weight, preferably from 0.3% to about 3% by weight, and preferably from about 0.5% to about 2% by weight with respect to the weight of the composition, including all ranges and subranges therebetween such as, for example, 0.4% to 1.5%, 1% to 3%, 0.3% to 2.5%, etc.

### Additional Additives

The composition of the invention can also comprise any additive usually used in the field under consideration. For example, dispersants such as poly(12-hydroxystearic acid), antioxidants, sunscreens, preserving agents, fragrances, fillers, neutralizing agents, cosmetic and dermatological active agents such as, for example, emollients, moisturizers, vitamins, essential fatty acids, viscosity increasing agents such as liposoluble/lipodispersible polymers, and mixtures thereof can be added. A non-exhaustive listing of such ingredients can be found in U.S. patent application publication no. 2004/0170586, the entire contents of which is hereby incorporated by reference. Further examples of suitable additional components can be found in the other references which have been incorporated by reference in this application. Still further examples of such additional ingredients may be found in the International Cosmetic Ingredient Dictionary and Handbook (9th ed. 2002).

A person skilled in the art will take care to select the optional additional additives and/or the amount thereof such that the advantageous properties of the composition according to the invention are not, or are not substantially adversely affected by the envisaged addition.

These substances may be selected variously by the person skilled in the art in order to prepare a composition which has the desired properties, for example, consistency or texture.

These additives may be present in the composition in a proportion from 0% to 99% (such as from 0.01% to 90%) relative to the total weight of the composition and further such as from 0.1% to 50% (if present), including all ranges and subranges therebetween.

Needless to say, the composition of the invention should be cosmetically or dermatologically acceptable, i.e., it should contain a non-toxic physiologically acceptable medium and should be able to be applied to the eyelashes of human beings.

According to preferred embodiments of the present invention, methods of treating, enhancing the appearance of, caring for and/or making up eyelashes by applying compositions of the present invention to the eyelashes in an amount sufficient to treat, enhance the appearance of, care for and/or make up the eyelashes are provided. Preferably, “making up” the eyelashes includes applying at least one coloring agent to the eyelashes in an amount sufficient to provide color to the eyelashes.

According to preferred embodiments of the present invention, methods of volumizing eyelashes comprising applying compositions of the present invention to eyelashes in an amount sufficient to volumize the eyelashes are provided.

In accordance with the preceding preferred embodiments, the compositions of the present invention are applied topically to the eyelashes in an amount sufficient to treat, enhance the appearance of, care for and/or make up the eyelashes. The compositions may be applied to the desired area as needed, preferably once or twice daily, more preferably once daily and then preferably allowed to dry before subjecting to contact such as with clothing or other objects (for example, a glass or a topcoat). Preferably, the composition is allowed to dry for about 1 minute or less, more preferably for about 45 seconds or less.

According to preferred embodiments of the present invention, methods of improving removal properties of an eyelash composition from eyelashes in visible pieces comprising combining at least one latex and at least one unalkoxylated wax in a weight ratio of unalkoxylated wax to latex (active ingredient) of 1.0 or less, and water during formation of the emulsion compositions such that the eyelash composition can be removed from eyelashes in visible pieces during removal are provided. Preferably, at least one aqueous phase film forming agent and/or at least one surfactant and/or at least one coloring agent and/or at least one water-soluble natural gum is also added during formation of the emulsion compositions. Preferably, the o/w emulsion compositions are mascara compositions which further comprise at least one coloring agent. Preferably, the o/w emulsion compositions contain minimal amounts of animal-derived ingredients or are free of such ingredients.

According to preferred embodiments of the present invention, methods of making oil-in-water (o/w) emulsion compositions which are capable of volumizing eyelashes and/or tubing after application are also provided. Preferably, the compositions possess sebum and/or water-resistance properties. Such methods comprise combining at least one latex and at least one unalkoxylated wax in a weight ratio of unalkoxylated wax to latex (active ingredient) of 1.0 or less, and water during formation of the emulsion compositions. Preferably, at least one aqueous phase film forming agent and/or at least one surfactant and/or at least one coloring agent and/or at least one water-soluble natural gum is also added during formation of the emulsion compositions. Preferably, the o/w emulsion compositions are eyelash compositions, in particular mascara compositions which further comprise at least one coloring agent. Preferably, the o/w emulsion compositions contain minimal amounts of animal-derived ingredients or are free of such ingredients.

The present invention also envisages kits and/or prepackaged materials suitable for consumer use containing one or more compositions according to the description herein, alone or in combination with makeup products such as basecoats, topcoats, removal compositions, etc. The packaging and application device for any subject of the invention may be chosen and manufactured by persons skilled in the art on the basis of their general knowledge, and adapted according to the nature of the composition to be packaged. Indeed, the type of device to be used can be in particular linked to the consistency of the composition, in particular to its viscosity; it can also depend on the nature of the constituents present in the composition, such as the presence of volatile compounds.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective measurements. The following examples are intended to illustrate the invention without limiting the scope as a result. The percentages are given on a weight basis.

### EXAMPLES

### Example 1: General O/W Emulsion Composition

| Ingredient | Amount | Preferred Amount |
|---|---|---|
| Wax | 1-25% | 6-12% |
| Latex (active ingredient) | 1-30% | 7.5-15% |
| Surfactant | 1-30% | 5-15% |
| Natural Gum | 0.1-20% | 1-5% |
| Aqueous phase Film | 0.1-10% | 0.5-5% |
| Former | | |
| Oil | 0-20% | 0-5% |
| Coloring Agent | 1-20% | 5-15% |
| Water | 10-60% | 25-50% |
| Other Ingredients (e.g., | 0-30% | 5-15% |
| monoalcohol, humectant, | | |
| preservative, filler, pH | | |
| adjuster, chelating agent) | | |

### Example 2: Sample O/W Emulsion Composition

| Ingredient | Amount |
|---|---|
| Unalkoxylated Wax | 10% |
| Latex (active ingredient) | 12% |
| Surfactant | 10% |
| Natural Gum | 3% |
| Aqueous phase Film Former | 1.5% |
| Ethanol | 6% |
| Polyol | 1% |
| Coloring Agent | 10% |
| Water | QS |
| Other Ingredients (e.g., preservative, | 1% |
| filler, pH adjuster, chelating agent) | |

### Example 3: Comparative Testing

An O/W emulsion mascara product of Example 2 (inventive/invention example) was compared to a commercial mascara product containing a weight ratio of unalkoxylated wax to latex (active ingredient) of >1.3 (comparative example).

The invention and comparative examples were tested on different eyelashes of the same subjects (females 18-50 years of age with medium to long eyelashes, no hooded eyelids), as follows:

The invention and comparative examples were each individually scooped inside the tube container, twisting and pumping slightly 3 times before removing from the tube. The wand tip was swiped against the rubber stopper of the mascara tube to remove excess product. Holding the wand parallel to the ground, a cosmetologist individually applied the comparative and invention examples on different upper eyelashes of a subject with a slight rotating motion the wand slightly extending to the tip of the lashes. The first 5 strokes starting from the outer corner of the eye at the root of the lashes, the second 5 strokes in the center of the lashes and the third strokes to the inner corner of the lashes for a total of 15 strokes The cosmetologist then re-dipped the mascara wand and repeated the procedure on the same eyelashes completing the application with a total of 30 strokes

Results: During application, the invention composition had slightly more immediate product deposit onto eyelashes, added length, spiking (lashes sticking together to provide a spiky look), and thickening than the comparative example. The invention composition also had noticeably more intensity than the comparative composition—the invention composition was much more visibly apparent on lashes than the comparative composition.

After six hours, both formulas performed at parity with respect to wear properties. Further, the compositions performed similarly with respect to removal using Garnier Micellar Water Pink Cap.

What is claimed is:

1. An oil-in-water (o/w) emulsion composition comprising water, at least one surfactant, at least one latex and at least one unalkoxylated wax, wherein the composition contains a weight ratio of unalkoxylated wax to latex (active ingredient) of 1.0 or less.

2. The composition of claim 1, wherein the unalkoxylated wax is synthetic beeswax.

3. The composition of claim 1, wherein the latex is a dispersion of polymer particles comprising at least one styrene group in an aqueous liquid phase, preferably particles of styrene/acrylates/ammonium methacrylate copolymer.

4. The composition of claim 1, further comprising at least one compound selected from the group consisting of coloring agents, water-soluble natural gums, aqueous phase film forming agents, and mixtures thereof.

5. The composition of claim 1, further comprising at least one coloring agent, preferably at least one pigment.

6. The composition of claim 1, further comprising at least one water-soluble natural gum, preferably acacia gum.

7. The composition of claim 1, further comprising at least one aqueous phase film forming agent, preferably a vinylpyrrolidone copolymer, preferably a vinylpyrrolidone/eicosene copolymer.

8. The composition of claim 1, wherein the composition is devoid of natural beeswax and/or paraffin wax.

9. The composition of claim 1, wherein the composition is devoid of animal-derived ingredients.

10. The composition of claim 1, further comprising from about 4% to about 8% of C2-C5 monoalcohol by weight with respect to the total weight of the composition, preferably ethanol.

11. The composition of claim 1, in the form of a mascara composition.

12. A method of making up eyelashes comprising applying the composition of claim 1 to eyelashes in an amount sufficient to makeup the eyelashes.

13. A method of volumizing eyelashes comprising applying the composition of claim 1 to eyelashes in an amount sufficient to volumize the eyelashes.

14. A method of making an oil-in-water (o/w) emulsion composition comprising combining water, at least one surfactant, at least one latex and at least one unalkoxylated wax, wherein the latex and unalkoxylated wax are in a weight ratio of unalkoxylated wax to latex (active ingredient) of 1.0 or less during formation of the emulsion compositions.
